(19) United States
(12) Patent Application Publication
(10) Pub. No.: US 2023/0406894 A1
(43) Pub. Date: Dec. 21, 2023

(54) PRODUCTION AND USE OF A FREEZE-DRIED OR READY-FOR-LYOPHILIZATION HUMAN RNASE INHIBITOR

(71) Applicant: BLIRT S.A., Gdansk (PL)

(72) Inventors: Agnieszka STANISLAWSKA, Gdansk (PL); Justyna LEIBNER-CISZAK, Borkowo (PL); Wioleta LADEMAN, Banino (PL); Monika WICKA-GROCHOCKA, Tczew (PL); Monika KOTKIEWICZ (nee Pokorska), Gdansk (PL); Robert BRODZIK, Gdansk (PL); Jan BALSEWICZ, Gdynia (PL); Arkadiusz POPINIGIS, Gdansk (PL); Dominik ZIETKOWSKI, Gdynia (PL); Krzysztof KUR, Reda (PL)

(21) Appl. No.: 18/035,143

(22) PCT Filed: Nov. 25, 2021
(86) PCT No.: PCT/PL2021/000085

§ 371 Date: May 3, 2023

(30) Foreign Application Priority Data
Nov. 25, 2020 (PL) P.436077

(51) Int. Cl. C07K 14/47

(52) U.S. Cl. CPC C07K 14/4703

(57) ABSTRACT

The subject of the invention is a freeze-dried and ready-for-lyophilization form of the recombinant human RNase inhibitor of SEQ ID NO 1 of the wild type variant in full size (50 kDa), which retains the properties of a recombinant human RNase inhibitor and can be used in the industrial applications including molecular biology and in vitro diagnostics. The developed formulation as well as the freeze-dried form of the recombinant human RNase inhibitor was successfully prepared for the first time in the full size variant. The subject of the invention is a freeze-dried variant of a recombinant human RNase inhibitor and the process of its production resulting in the formation of a powdered form of an RNase inhibitor, which maintains its functionality and increases its stability. Additionally, a dedicated liquid formulation ready for the freeze-drying process was developed. The subject of the invention is the opportunity of using both forms described above for diagnostics in vitro and molecular biology in the processes and kits for the isolation and purification of RNA genetic material (ribonucleic acid), systems for cDNA strand synthesis and RT-PCR, RT-qPCR, RT-LAMP techniques, as well as kits for transcription and translation in vitro and dedicated solutions for RNA amplification.

The subject of the invention is a freeze-dried form and ready-to-lyophilization formulation of a wild type recombinant human RNase inhibitor with SEQ.1 for applications in molecular biology and in vitro diagnostics. The developed formulation as well as the freeze-dried form of the recombinant human RNase inhibitor was successfully prepared for the first time in the full variant with a size of approx. 50 kDa. The subject of the invention is a freeze-dried variant of a recombinant human RNase inhibitor and the process of its production resulting in the formation of a powdered form of an RNase inhibitor, which maintains its functionality and increases its stability. Additionally, a dedicated liquid formulation ready for the freeze-drying process was developed. The subject of the invention is the opportunity of using both forms described above for diagnostics in vitro and molecular biology in the processes and kits for the isolation and purification of RNA genetic material (ribonucleic acid), systems for cDNA strand synthesis and RT-PCR, RT-qPCR, RT-LAMP techniques, as well as kits for transcription and translation in vitro and dedicated solutions for RNA amplification.

A recombinant RNase inhibitor is a protein purified from E. coli carrying a plasmid with the cloned gene encoding a human RNase inhibitor. It inhibits the activity of ribonuclease such as RNase A, RNase B, RNase C by non-covalent binding in a 1:1 ratio. It has no activity against RNase 1, RNase T1, RNase T2, S1 nuclease and RNase H. It completely inhibits the activity of RNase A, B and C, does not inhibit the activity of polymerases and reverse transcriptases commonly used for amplification and detection of RNA material. The purified RNase inhibitor in the recombinant variant is free of DNases, RNases and protease contamination and stabilizes RNA at temperatures up to 60° C. It is stable in a wide range of temperatures, pH and DTT concentrations.

The format of the freeze-dried (water being removed by means of sublimation from frozen material) diagnostic tests that enable genetic analysis of nucleic acids has significant competitive advantages in terms of the possibility of storage at room temperature, not refrigerated, extended shelf life and lower transport costs (no cooling/freezing costs). Moreover, the freeze-dried form of the powder is lighter and takes up a smaller volume compared to liquid/dissolved solutions. The present invention is applicable to all diagnostic assays based on RNA isolation, amplification, modification and detection.

In conventional formulations of diagnostic kits based on nucleic acid amplification techniques (including PCR, qPCR, RT-PCR, RT-qPCR, RT-LAMP, RCA), the enzymes used there are in a formulation containing glycerol (up to 50%), which is a cryoprotectant commonly used to stabilize proteins, enzymes and mixes used in biology and molecular diagnostics. But even a concentration of this compound greater than 0.5% in an enzyme or protein preparation may hinder or prevent the lyophilization process, which means that the currently used variants cannot be used in the ever faster growing segment of enzymes/proteins compatible with lyophilization (lyo-ready). The formulation of the present protein (RNase inhibitor) in the lyo-ready version does not contain this compound and other similarly active additives, so that it can be used on a larger scale in the increasingly promoted, freeze-dried diagnostic kits or as a single product (freeze-dried protein) or in reaction mixtures composed by the target buyer [e.g. manufacturer of a diagnostic test that includes an RNase inhibitor]. Such tests are gaining more and more importance in the dynamically developing market of POCT diagnostics (Point Of Care Testing). In both health and agriculture, rapid detection of infectious diseases at the test site can reduce response times while increasing treatment efficacy and survival. Rapid detection of plant pathogens can also reduce unnecessary pesticide use. The same concept applies to healthcare, where basic diagnostic tests with the use of freeze-dried mixtures for RT-qPCR, RT-LAMP or RCA can increase the effectiveness of the treatment of infectious diseases.

There are currently two known entities that offer a powdered/freeze-dried human inhibitor—these are MyBioSource, Inc. (USA) and Wuhan USCN Business Co., Ltd. (China) with catalog numbers of the products MBS2018646 and RPB444Hu01 respectively. However, the freeze-dried protein that they offer is smaller by approx. 44% (approx. 28 kDa, Asp218˜Glu444 fragment), and thus shortened by almost a half, which does not guarantee effective blocking/inhibition of RNase due to the fact that the mechanism of action of the RNase inhibitor is based on the non-covalent linkage of this protein to the RNase active site. The molecule truncated by almost half is ineffective at blocking the active site of RNA-degrading enzymes, as confirmed by an experiment conducted by Blirt. In addition, the manufacturer of this preparation has not verified whether the protein is effective in blocking RNases, proposing its main application as a reaction control in protein detection techniques.

Human RNase inhibitor isolated from the human placenta (Blackburn et al., J. Biol. Chem., 252, 5904 (1977), Lee et al. Biochem., 28, 219 (1988), Lee et al. Biochem., 28, 225 (1989)) or in a recombinant variant, it is a product widely used in diagnostics and research in which the analyzed material is RNA. It is used to inactivate common enzymes (ribonuclease/RNases) that cause irreversible degradation of RNA. The RNase inhibitor protects the genetic material from damage by the action of common exogenous RNases, thus enabling correct diagnosis and reducing the risk of unwanted artifacts (false negative results). Currently, it is a particularly important tool due to the fact that this protein is one of the components of diagnostic kits detecting the presence of RNA genetic material of viruses, including SARS-CoV-2. These kits are based on the isolation of the genetic material of the virus (RNA of the virus) from a swab sample from the patient, and then performing a reverse transcription reaction (rewriting information from RNA into cDNA) and amplification using a very sensitive detection method (including RT-qPCR or RT-LAMP).

The RNA molecule is by nature very fragile and extremely sensitive to the presence of common RNases. Only the use of an additional protein—an RNase inhibitor allows for the protection of extremely valuable diagnostically valuable material, i.e. virus RNA isolated from a swab sample. It is a guarantee of obtaining reliable and repeatable results. Omission of the addition of an RNase inhibitor may result in a false negative result of the diagnostic reaction, as a result of the degradation of the free RNA of the virus (under the influence of RNases), and thus giving a false result and incorrect determination of a patient infected with e.g. SARS-CoV-2 virus as a healthy person.

Recombinant RNase inhibitor in lyo-ready form, thanks to the use of an appropriate production method and a specially composed formulation buffer, shows increased thermostability. This allows for safer transport of the protein preparation, but most of all, it makes it less sensitive to the loss of its functionality during lyophilization (freeze-drying). On the other hand, an RNase inhibitor in a powdered (freeze-dried) form shows the highest thermostability and stability of all known forms of this protein. This allows for much safer transport and long-term storage of protein, even in non-refrigerated conditions. In addition, the reduction in volume and weight in the freeze-drying process generates economic and environmental benefits in terms of transport and storage.

The aim of the present invention was to obtain a recombinant RNase inhibitor in full size (50 kDa) in the form of a lyophilisate (powder) and in a liquid form ready for the lyophilization process. Thanks to the use of an appropriate production method and a specially composed formulation buffer, both freeze dried and ready-for-lyophilization forms show increased thermostability. This allows for easier and safer transport of protein (both freeze dried and ready-for-lyophilization forms) and long-term storage of the protein in powdered form under non-refrigerated conditions.

The subject of the invention is a freeze-dried and ready-for-lyophilization form of the recombinant human RNase inhibitor of the wild type variant in full size (50 kDa), which retains the properties of a recombinant human RNase inhibitor stored under refrigeration in liquid formulations known from the state of the art, regardless of whether they are simple or containing various types of stabilizers, and is characterized by increased thermal stability.

Freeze-dried form of recombinant human RNase inhibitor of the wild-type variant wherein:
a) the lyophilisate contains a wild-type variant of recombinant human RNase inhibitor having the sequence of SEQ Id No 1,
b) it retains its properties after the incubation period at 60° C. for min. 5 hours and at 65° C. for min. 2 hours and at 70° C. for mM. 30 min

Lyophilization-ready recombinant human RNase inhibitor of the wild-type variant that:
a) contains a recombinant human RNase inhibitor of the wild-type variant having the sequence of SEQ ID No 1,
b) retains its properties after the incubation period at 50° C. for 6 hours (similar to the glycerol version),
c) contains a buffer of pH 7.0-8.2 in a concentration between 5 mM and 50 mM,
d) contains a monovalent salt in a concentration between 5 mM and 100 mM,
e) contains a reducing agent in a concentration between 1 mM and 20 mM,
f) does not contain glycerol.

Freeze-dried form of the recombinant human RNase inhibitor of the wild-type variant having the sequence of o SEQ ID No 1 suitable for transport under non-refrigerated conditions, retaining the functionality and properties of a recombinant human RNase inhibitor as in liquid formulations known in the art.

A method of obtaining a freeze-dried, full-size (50 kDa) recombinant human RNase inhibitor of the wild-type variant having the amino acid sequence of SEQ ID No 1, wherein:
a) the freezing temperature during the preparation of samples for lyophilization is in the range from −20° C. to −195.8° C.,
b) during the preparation of samples for lyophilization, the thermal treatment is not longer than 5 hours,
c) the samples are freeze-dried not longer than 63 h,
d) the vacuum used in the process is not more than 0.05 mbar.

Freeze-dried and/or ready-for-lyophilization, full-size (50 kDa) recombinant human RNase inhibitor of the wild-type variant having the sequence of SEQ ID No 1 in kits for cDNA synthesis, RT-PCR, RT-qPCR, RT-LAMP and NGS in the freeze-dried form.

Freeze-dried and/or ready-for-lyophilization, full-size (50 kDa) recombinant human RNase inhibitor of the wild-type variant having the sequence of SEQ ID No 1 for the isolation and purification of RNA genetic material from any source (reaction mixtures, cells, cell lyophilisates, etc.).

Freeze-dried and/or ready-for-lyophilization, full-size (50 kDa) recombinant human RNase inhibitor of the wild-type variant having the sequence of SEQ ID No 1 in RNA sequence amplification processes in industrial applications.

Freeze-dried and/or ready-for-lyophilization, full-size (50 kDa) recombinant human RNase inhibitor of the wild-type variant having the sequence of SEQ ID No 1 in transcription and translation processes in vitro and dedicated kits/reagents.

The terms used above and in the specification and claims have the following meanings:
Human RNase Inhibitor—a recombinant protein of the invention with the sequence SEQ ID No 1.
Powdered form—stands for a freeze-dried form.
Form ready-for-lyophilization—indicates liquid form.

### DESCRIPTION OF FIGURES, TABLES AND SEQUENCES

FIG. 1: The results of the electrophoretic separation of RNA protected by a recombinant human RNase inhibitor having the sequence of SEQ ID No 1 in a formulation ready-for-lyophilization at two different concentrations (1.40 U/μL and 2.160 U/μL with +/−controls) after storage at 37° C. for 4 weeks. A control RNase inhibitor (T) known in the art contained glycerol in the formulation and was stored in accordance with the instructions for use at −20° C. The results confirm that the RNase inhibitor in the formulation ready-for-lyophilization retains its RNA protection properties after storage at 37° C. for a minimum period of 4 weeks.

FIG. 2: The results of the functionality of RNase inhibitor having the sequence of SEQ ID No 1 in all tested variants of the ready-for-lyophilization formulation supplemented with various additives. The results show that the following additives can be added to the basic formulation of the lyophilization buffer in the tested range without loss of functionality: phenylalanine (up to 0.5%), Ficoll (up to 0.5%), sucrose (up to 0.5%), Tween20 (up to 0.05%), mannitol (up to 0.5%), sorbitol (up to 0.5%).

FIG. 3: Results of electrophoretic separation of RNA protected by a recombinant human RNase inhibitor having the sequence of SEQ ID No 1 after storage at 50° C. for 6 hours (with +/−controls). The result for two independent production batches of an RNase inhibitor ready-for-lyophilization (RT35L) and for an RNase inhibitor containing glycerol in the formulation buffer (RT35). The results confirm that both formulations retain their RNA protection properties after storage at 50° C. for a minimum period of 6 hours.

FIG. 4: Results of the electrophoretic separation of RNA protected by a recombinant human RNase inhibitor having the sequence of SEQ ID No 1 in a ready-to-lyophilization faun containing glycerol in combination with a freeze-dried fragment of a human RNase inhibitor (approx. 28 kDa, fragment Asp218-Glu444). A—Recombinant human RNase inhibitor having the sequence of SEQ ID No 1 in formulation with glycerol; B—Dissolved powder of the recombinant fragment of the RNase inhibitor RPB444Hu01; C—Recombinant human RNase inhibitor having the sequence of SEQ ID No 1 in formulation ready-for-lyophilization (without glycerol). The results confirm that the truncated version of the RNase inhibitor is ineffective in blocking the RNase A active site.

FIG. 5. Results of electrophoretic separation of RNA protected by a recombinant human RNase inhibitor with SEQ ID No 1 sequence after storage at 70° C. for 30 minutes (with +/−controls) as lyophilisates (lanes 1-4) compared to a formulation of this protein containing glycerol (RT35). The results confirm the increased thermostability of the freeze-dried form.

FIG. 6. Results of electrophoretic separation of RNA protected by a recombinant human RNase inhibitor with SEQ ID No 1 sequence after storage at 65° C. for 2 hours (with +/−controls) as lyophilisates (lanes B1-4) compared to a formulation of this protein containing glycerol (B5). The results confirm the increased thermostability of the freeze-dried form.

FIG. 7. An example of the appearance of a freeze-dried RNase inhibitor of SEQ ID No 1.

FIG. 8. The use of a human RNase inhibitor having the sequence SEQ ID No 1 in the form ready for lyophilization in the RT-LAMP kit for the detection of RNA templates at various concentrations before and after the lyophilization process and its influence on the RT-LAMP reaction.

FIG. 9 A form of a freeze-dried RNase Inhibitor having the sequence SEQ ID No.1 obtained using the full non-constant temperature and pressure profile.

FIG. 10. The results of the functionality of RNase Inhibitor having the sequence of SEQ ID No 1 in all tested variants of the ready-for-lyophilization formulation supplemented with various additives and lyophilized using the full non-constant temperature and pressure profile. The results show that the following additives as well as the thermal and pressure conditions can be implemented without the loss of functionality.

Table 1. The content of the samples tested in example 1.
Table 2. Concentrations of additives to assess the effect on the functionality and stability of an RNase inhibitor having the sequence SEQ ID No 1 in a ready-for-lyophilization formulation buffer.
Table 3. The content of the reaction mixtures of the samples tested in Example 2
Table 4. The content of the reaction mixtures of the samples tested in Example 3.
Table 5. The content of the samples tested in example 4.
Table 6. Results of the dependence of Ct on the activity of proteins in the RT-LAMP kit, including the RNase inhibitor before and after lyophilization.
Table 7. Compositions of freeze-dried samples.
Table 8. Temperature and pressure conditions of the lyophilization profile.
Table 9. The content of the reaction mixtures of the samples test in Example 7.

SEQ ID No 1—represents the amino acid sequence of the human RNase inhibitor protein of the wild-type variant.

The invention is illustrated by the following non-limiting examples.

### EXAMPLE 1

### Preparation of a Ready-To-Lyophilization Formulation of a Recombinant Human RNase Inhibitor of the Wild-Type Variant having the Sequence SEQ ID No 1 and Study of its Thermal Stability in this Formulation

The thermal stability of the ready-for-lyophilization formulation was verified compared to the standard formulation suspended in a formulation buffer containing glycerol. The composition of the ready-for-lyophilization formulation (lyo-ready) contains 20 mM HEPES-KOH (pH 7.6); 50 mM KCl and 8 mM reducing agent.

The thermal stability of the ready-for-lyophilization formulation (obtained according to example 1) was tested in comparison to the standard form suspended in a formulation buffer containing glycerol. RNase inhibitor samples at two concentrations (1.40 U/μL and 2.160 U/μL) in the form ready-for-lyophilization, without glycerol, were incubated for 4 weeks at 37° C., and then the activity of the formulation was determined. The control of the reaction and, at the same time, the reference was an RNase inhibitor known in the art (T), stored according to the manufacturer's instructions, at −20° C. The negative control of the reaction and pure RNA without the addition of an RNase inhibitor and RNase A, while the positive control of the reaction—an RNA sample treated with RNase A without the RNase inhibitor.

TABLE 1

| | RNase inhibitor | RNase A | RNA |
|---|---|---|---|
| 1. Lyo-ready RNase | + | + | + |
| Inhibitor 40 U/μL | | | |
| 2. Lyo-ready | + | + | + |
| RNase Inhibitor | | | |
| 160 U/μL | | | |
| T—RNase inhibitor known in | + | + | + |
| the art 40 U/μL | | | |
| K+ | − | + | + |
| K− | − | − | + |

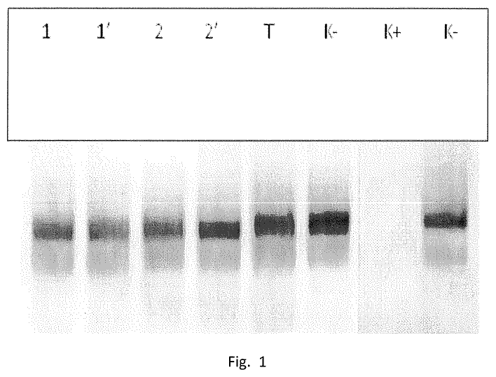

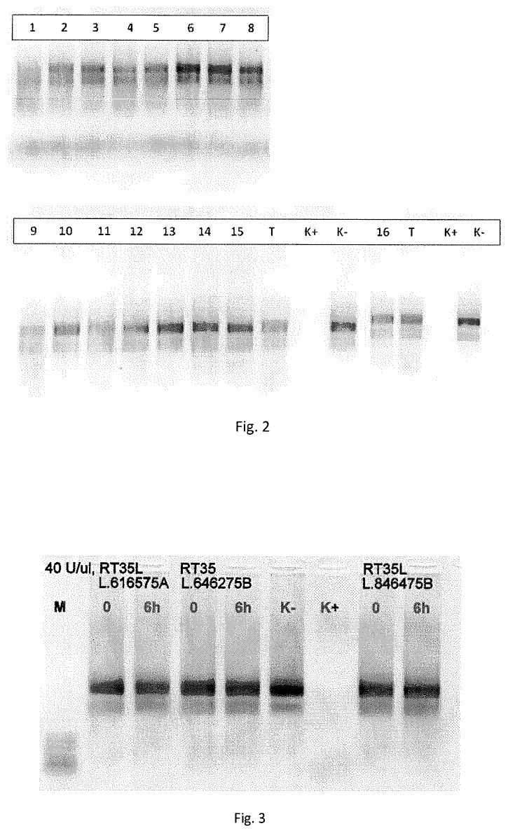

5 U of each RNase inhibitor test sample (40 U/μL) was taken and incubated with 1.5 ng of RNase A for 10 minutes at room temperature. Then 2 μg of RNA was added to each of them and incubated for 15 minutes at 37° C. To visualize the result of the RNase inhibitor activity assay, agarose electrophoresis of the samples was performed at 125 V for 40 minutes with ethidium bromide. The collective results are presented in FIG. 1.

The study confirmed that the RNase inhibitor in the proposed ready-for-lyophilization formulation shows full thermal stability for a minimum period of 4 weeks at 37° C. The same result is obtained for the RNase inhibitor in the standard version with 50% glycerol in the BURT formulation buffer (RT35), which distinguishes it from other available RNase inhibitors from other manufacturers and suppliers.

### EXAMPLE 2

### Optimization Studies for POSSIBLE RECOMBINANT HUMAN RNASE INHIBITOR Formulations of the Wild-Type Variant having the Sequence of a SEQ ID No 1, ready-for-lyophilization

In addition, the ready-for-lyophilization formulation was optimized without the need to supplement additives and with the use of the most commonly used lyophilization additives. For this purpose, a series of freeze-drying reactions were carried out in the presence of various reaction components, in their different concentrations and its interactions. For this purpose, various combinations of the components of the formulation were tested, and then the inhibitor samples were freeze-dried:

TABLE 2

| | Phenylalanine | Ficoll | Sucrose | Tween 20 |
|---|---|---|---|---|
| 1 | — | — | — | — |
| 2 | — | — | | 0.05% |
| 3 | 0.5% | 0.5% | | 0.05% |
| 4 | 0.2% | 0.5% | | 0.05% |
| 5 | 0.1% | 0.5% | | 0.05% |
| 6 | 0.5% | — | 0.5% | 0.05% |
| 7 | 0.2% | — | 0.5% | 0.05% |
| 8 | 0.1% | — | 0.5% | |
| | Mannitol | Sorbitol | Sucrose | Tween 20 |
| 9 | — | — | — | — |
| 10 | — | — | | 0.05% |
| 11 | — | — | 0.5% | — |
| 12 | — | — | 0.5% | 0.05% |
| 13 | 0.5% | — | 0.2% | 0.05% |
| 14 | 0.2% | — | 0.5% | 0.05% |
| 15 | 0.5% | 0.2% | — | 0.05% |
| 16 | 0.2% | 0.5% | — | 0.05% |

Then the freeze-dried samples were dissolved in DEPC water and their activity was verified according to the method described above

TABLE 3

| | RNase inhibitor | RNase A | RNA |
|---|---|---|---|
| 1-16 freeze-dried | + | + | + |
| samples, and then | | | |
| dissolved to 40 U/μL | | | |
| T—RNase | + | + | + |
| inhibitor purchased | | | |
| from competitors 40 | | | |
| U/μL | | | |
| K+ | − | + | + |
| K− | − | − | + |

The proposed ready-for-lyophilization formulation of a recombinant RNase inhibitor allows for its universal use in lyophilization processes, with the use of various lyophilization additives, depending on the recipient's needs. After dissolving the freeze-dried powder, the RNase inhibitor shows full functionality in all tested samples, which confirms the possibility of its universal and safe use.

### EXAMPLE 3

### Stability Studies of Ready-For-Lyophilization Recombinant Human RNase Inhibitor having the Sequence of SEQ ID No 1

RNase inhibitor samples from two independent production processes (1. L616575A and 2. L846475B) in a ready-to-lyophilization form at a concentration of 40 U/ΞL, was incubated for 6 hours at 50 ° C., and then the functionality of the preparations was determined. The reference was an RNase inhibitor in the standard liquid version, containing glycerol in the formulation buffer (L646275B), subjected to the same incubation for 6 h at 50° C., as the variant ready-for-lyophilization. The negative control of the reaction was pure RNA without the addition of an RNase inhibitor and RNase A, while the positive control of the reaction was a RNase sample treated with RNase without an RNase inhibitor.

TABLE 4

| | RNase inhibitor | RNase A | RNA |
|---|---|---|---|
| RNase inhibitor | + | + | + |
| Lyo-ready 40 U/μL | | | |
| L616575A | | | |
| RNase inhibitor 40 U/μL | + | + | + |
| L646275B | | | |
| K+ | − | + | + |
| K− | − | − | + |

40 U of each RNase inhibitor test sample (40 U/μL) was taken and incubated with 5 ng RNase A for 10 minutes at RT. Then 1 μg of RNA was added to each of them and incubated for 15 min at 37° C. To visualize the results of the RNase inhibitor functionality assay, agarose electrophoresis of the samples was performed at 125 V for 40 minutes with ethidium bromide. The results of the electrophoretic separation are presented in FIG. 3.

The study confirms that the RNase inhibitor having the sequence of SEQ Id No 1, ready-for-lyophilization (without the addition of glycerol in the formulation buffer) retains full functionality after incubation for 6 h at 50° C. An identical result was obtained for the RNase inhibitor standard version with 50% glycerol in BURT formulation buffer, catalog number RT35.

### EXAMPLE 4

### Comparative Study of a Ready-For-Lyophilization Formulation of a Recombinant Human RNase Inhibitor having the Sequence of SEQ ID No 1 with a Fragment of a Human RNase Inhibitor (approx. 28 kDa, fragment Asp218-Glu444) Known in the art, in a Freeze-Dried Form

Test product: Recombinant Ribonuclease Inhibitor (RI), Cat. No. RPB444Hu01, 10 μg of the powder was suspended in 100 μL of sterile DEPC water, giving a concentration of 0.1 μg/μL. The functionality of the preparation was verified in comparison to the liquid RNase inhibitor in the version ready-for-lyophilization and in the formulation buffer containing glycerol.

The negative control of the reaction was pure RNA without the addition of an RNase inhibitor and RNase A, while the positive control of the reaction was a RNA sample treated with RNase A without the RNase inhibitor.

TABLE 5

| | RNase inhibitor | RNase A | RNA |
|---|---|---|---|
| .A. RNase inhibitor | + | + | + |
| version with | | | |
| glycerol | | | |
| B. Recombinant | + | + | + |
| Ribonuclease | | | |
| Inhibitor (RI), | | | |
| Solubilized powder, | | | |
| Cat. No. | | | |
| RPB444Hu01 | | | |
| .C. RNase inhibitor | + | + | + |
| Lyo-ready | | | |
| K+ | − | + | + |
| K− | − | − | + |

The same amount of protein for the reaction was taken, i.e. 10 mL of dissolved powder (1 μg) and 40 U (approx. 1 μg) RNase inhibitor reference samples (lyo-ready liquid version and glycerol version in formulation buffer) and incubated with 5 ng RNase A for 10 minutes at RT. Then 1 μg of RNA was added to each of them and incubated for 15 min at 37° C.

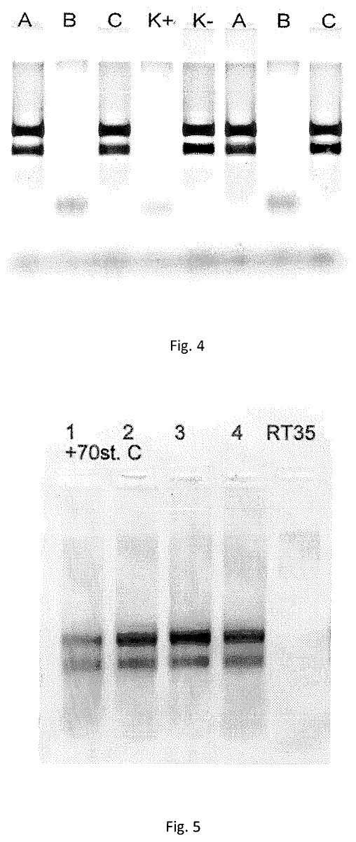

To visualize the results of the RNase inhibitor functionality assay, agarose electrophoresis of the samples was performed at 125 V for 40 minutes with ethidium bromide. The results of the electrophoretic separation are presented in FIG. 4. Recombinant human RNase inhibitor having the sequence of SEQ Id No 1 in the formulation with and without glycerol functions properly protecting RNA against the action of RNase, on the other hand, a fragment of RNase (truncated version) does not protect RNA from the action of RNase.

### EXAMPLE 5

### Study of the Stability of the Lyophilised Powders of the Recombinant Human RNase Inhibitor having the Sequence of SEQ ID No 1 in Comparison to the Formulation of the Recombinant Human RNase Inhibitor of SEQ ID No 1 in the Formulation with Glycerol under Various Temperature Conditions

Samples of the freeze-dried RNase inhibitor (1-4) and its liquid counterpart in a formulation buffer containing glycerol (40 U/μl) were incubated for 30 min. at 70° C., and for 2 hours at 65° C. Subsequently, the freeze-dried samples were dissolved in DEPC water and their functionality was verified in comparison to the liquid RNase inhibitor.

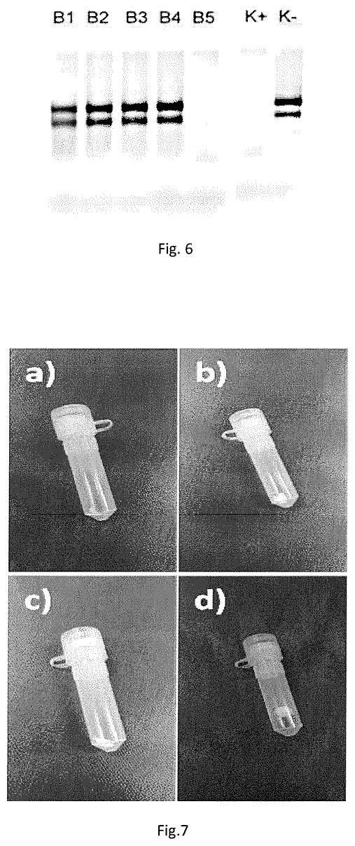

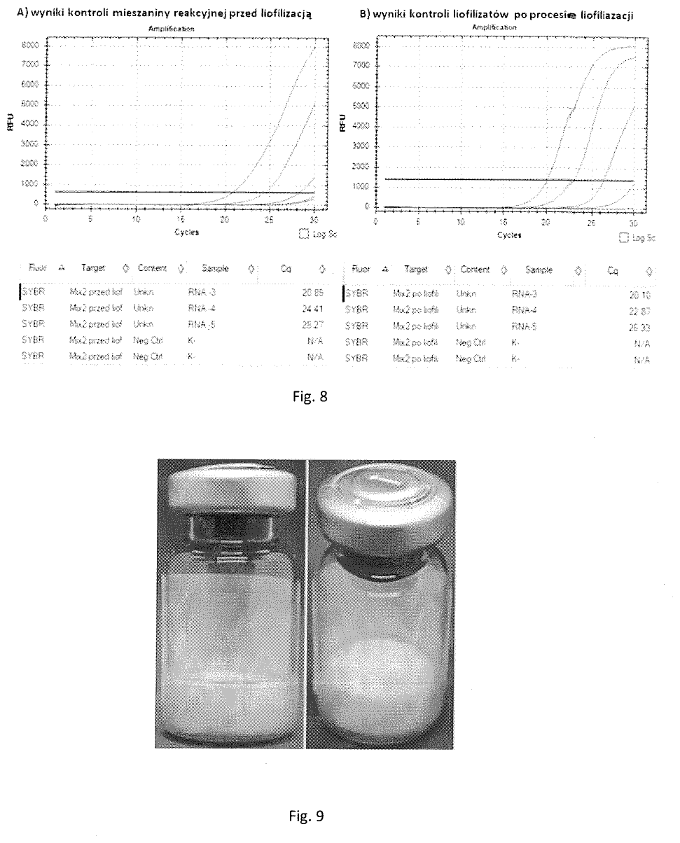

For this purpose, 40 U of each RNase inhibitor test sample (liquid version and dissolved powder) were taken and incubated with 5 ng of RNase A for 10 min at room temperature. Then 1 μg of RNA was added to each of them and incubated for 15 min at 37° C. To visualize the results of the RNase inhibitor functionality assay, agarose electrophoresis of the samples was performed at 125 V for 40 minutes with ethidium bromide. The results of the electrophoretic separation are presented in FIG. 5. (for 70° C. for 30 minutes) and FIG. 6 (for 65° C. for 2 hours). Recombinant human RNase inhibitor having the sequence of SEQ ID No. 1, with glycerol, loses activity after incubation at 70° C. for 30 min. In contrast, a recombinant human RNase inhibitor having the sequence of SEQ ID No 1 in a freeze-dried form is active after incubation at 70° C. for a minimum of 30 min. Similar results were obtained for the temperature of 65° C., respectively, for the period of 2 hours.

### EXAMPLE 6

Preparation of a Freeze-Dried Recombinant Human RNase Inhibitor of the Wild-Type Variant, having the Sequence of SEQ ID No 1

In order to obtain a freeze-dried recombinant RNase inhibitor of the wild-type variant, having the sequence of SEQ Id No 1, inhibitor preparations in the ready-to-lyophilization formulation from Example 1, without glycerol content, but containing selected and optimized lyophilization additives (so-called cryo- and lyo-protectants) from Example 2 were placed in 2 mL tubes, frozen to the temperature of −80° C. for 30 min, and then freeze-dried in the following conditions: manual mode, without heating the shelves, heating the pump for 20 min, freeze drying/drying: pressure 0.02 mbar, time: 12 hours. As a result of the freeze-drying process, freeze-dried powder preparations containing the RNase inhibitor SEQ ID No 1 were obtained. These lyophilisates differed in appearance depending on the additives used (cryo-/lyo-protectants), FIG. 7.

### EXAMPLE 7

### Example of using Ready-For-Lyophilization Formulation of the Variant Recombinant Human RNase Inhibitor Formulation with Sequence SEQ ID No 1 in RT-LAMP Kit

In order to determine the stability and functionality of an RNase inhibitor with sequence SEQ ID No 1 ready-for-lyophilization, a reaction mixture with three proteins was prepared in the RT-LAMP reaction mixture: reverse transcriptase, RNase inhibitor and Bst polymerase. The mixture was subjected to a single-step reverse transcription reaction and isothermal amplification according to the profile: temperature 65° C., time 60 s, X-fluorescence measurement, number of cycles 30.

Then the reaction mixture was made up to 52 μL with water and freeze-dried in the following conditions: manual mode, without heating the shelves, heating the pump for 20 min, freeze drying/basic drying: pressure 0.01 mbar, time: 15 h.

After the lyophilization process, the obtained lyophilisates were subjected to quality control with templates (3 serial dilutions of RNA) as for the control of the reaction mixture before freeze-drying. The collective results are presented in FIG. No. 8 A and B:

TABLE 6

| Template | Ct before lyophilization | Ct after lyophilization |
|---|---|---|
| RNA 10⁻³ | 20.9 | 20.1 |
| RNA 10⁻⁴ | 24.4 | 22.9 |
| RNA 10⁻⁵ | 28.3 | 26.3 |
| K− | − | − |
| K− | − | − |

The conducted studies indicate that freeze-dried proteins (including the RNase inhibitor ready-for-lyophilization) in the RT-LAMP reaction mixture retain their activity and functionality, do not lead to the formation of non-specific products and are stable. They can be successfully freeze-dried and used in sets for isothermal RNA amplification in the RT-LAMP system.

### EXAMPLE 8

### Preparation of the Full Temperature and Pressure Profile of the Freeze-Drying Process Recombinant Human RNase Inhibitor of the Wild-Type Variant, having the Sequence of SEQ ID No 1

In order to ensure the stability of the finished product, lyophilization conditions were selected that will not lead to a change in the protein functionality of the preparation and at the same time provide the appropriate atmosphere for storing the product. In contrast to the previous versions of the freeze-drying, the aim of this example was to create a lyophilization method with a full non-constant temperature and pressure profile in order to optimize the process in terms of quality and economy.

The effectiveness of the selection of the temperature and pressure profile during lyophilization was tested on 6 preparations of different compositions, which can be found in Table 7.

The prepared samples were lyophilized according to the profile described in Table 8.

TABLE 7

| | Excipient 1 | Excipient 2 | Active |
|---|---|---|---|
| Formulation | (Conc) | (Conc) | Ingredient |
| 1 | dextran (6%) | — | Lyo- |
| 2 | dextran (4%) | trehelose (2%) | ready |
| 3 | dextran (4%) | raffinose (2%) | RNase |
| 4 | Ficoll (6%) | — | Inhibitor |
| 5 | Ficoll (4%) | trehelose (2%) | 100 U/μl |
| 6 | Ficoll (4%) | raffinose (2%) | |

TABLE 8

| | | shelf | | | |
|---|---|---|---|---|---|
| | | temperature | time | pressure | |
| Steps | | [° C.] | [min] | [μbar] | description |
| thermal | loading | 5 | n/a | atmospheric | hold |
| treatment stage | 1 | −30 | 70 | atmospheric | ramp |
| | 2 | −30 | 180 | atmospheric | hold |
| primary | 3 | −30 | 15 | 50 | hold |
| drying stage | 4 | −30 | 4800 | 150 | hold |
| secondary | 5 | 20 | 180 | 50 | ramp |
| drying stage | 6 | 20 | 480 | 50 | hold |
| actions at | — | 20 | n/a | atmospheric | backfill |
| end of cycle | | | | | with N₂ |

| | |
|---|---|
| checkpoints/ | freeze temperature [° C.]: −55 |
| setpoints** | condenser setpoint [° C.]: −60 |
| | additional freeze [min]: 0 |
| | vacuum setpoint [μbar]: 500 |

Vials were filled with each preparation to a volume of 200 μl. The final RNase Inhibitor concentration in each of the proposed formulations was 50 U/μl.

The ingredients of the ready-for-lyophilization formulation contains 20 mM HEPES-KOH (pH 7.6), 50 mM KCl and 8 mM reducing agent

After freeze drying was complete, the samples were deposited and then screened for their functionality.

The freeze-dried samples were dissolved in DEPC water and their activity was verified according to the method described below.

TABLE 9

| | RNase Inhibitor | RNaseA | RNA |
|---|---|---|---|
| 1-6 freeze-dried | + | + | + |
| samples, and then | | | |
| dissolved to 40 U/μl | | | |
| K+ | − | + | + |
| K− | − | − | + |

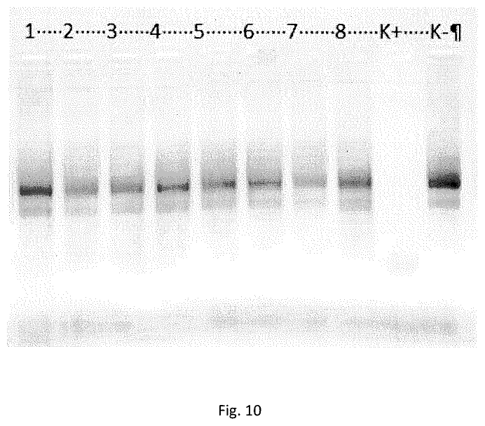

5 U of each RNase Inhibitor test sample was taken and incubated with 1.5 ng of RNase A for 10 minutes at room temperature. Then 2 μg of RNA was added to each of them and incubated for 15 minutes at 37° C. To visualize the result of the RNase Inhibitor activity assay, agarose electrophoresis of the sample was performed at 125 V for 40 minutes with ethidium bromide. The collective results are presented in FIG. 9-10.

The results show that the following additives as well as the full non-constant temperature and pressure conditions can be implemented without the loss of the functionality.

1-8. (canceled)

9. A liquid formulation comprising:
a protein having an amino acid sequence according to SEQ ID NO: 1;
a buffer of pH 7.0 to 8.2 in a concentration between 5 mM and 50 mM; and
a monovalent salt in a concentration between 5 mM and 100 mM; and
no glycerol.

10. The liquid formulation according to claim 9, wherein the buffer is 2-[4-(2-Hydroxyethyl)piperazin-1-yl]ethane-1-sulfonic acid (HEPES) adjusted with potassium hydroxide.

11. The liquid formulation according to claim 9, wherein the monovalent salt is potassium chloride.

12. A method for obtaining a protein powder, the method comprising freeze-drying the liquid formulation according to claim 9.

13. The method according to claim 12, wherein the liquid formulation is freeze-dried for a period of 63 hours or less.

14. The method according to claim 12, wherein the liquid formulation is freeze-dried under vacuum conditions not exceeding 0.05 mbar.

15. A protein powder produced according to the method of claim 12.


SEQUENCE LISTING (1 sequence)

SEQ ID NO: 1 - protein, 460 aa, Homo sapiens [sequence omitted]